(19) United States
(12) Patent Application Publication
(10) Pub. No.: US 2026/0283927 A1
(43) Pub. Date: Sep. 24, 2026

(54) CRYSTALLINE FORM OF CERAMIDE NP AND COMPOSITION INCLUDING THE SAME

(71) Applicant: CRODA KOREA LTD., Iksan-si, Jeonbuk-do (KR)

(72) Inventors: A Ra KIM, Iksan-si, Jeonbuk-do (KR); Hyun Cheol JANG, Iksan-si, Jeonbuk-do (KR); Young Lae YANG, Iksan-si, Jeonbuk-do (KR); Sang Chul KIM, Iksan-si, Jeonbuk-do (KR); Seung Won PARK, Iksan-si, Jeonbuk-do (KR)

(73) Assignee: CRODA KOREA LTD., Iksan-si, Jeonbuk-do (KR)

(21) Appl. No.: 19/469,933

(22) PCT Filed: Apr. 11, 2024
(86) PCT No.: PCT/KR2024/004852

§ 371 Date: Sep. 26, 2025

(30) Foreign Application Priority Data
Apr. 11, 2023 (KR) 10-2023-0047532

(51) Int. Cl. A61K 8/68; A61Q 19/00; C07C 233/18

(52) U.S. Cl. CPC A61K 8/68; A61Q 19/00; C07C 233/18; C07B 2200/13

(57) ABSTRACT

Provided are a novel crystalline form of ceramide NP and a composition including the same. A ceramide NP crystalline powder and a composition including the same, according to an aspect, have excellent stability and thus, can be useful for compositions, for example, a cosmetic composition.

### CROSS REFERENCE TO RELATED APPLICATIONS

This application is the U.S. National Phase of PCT International Application No. PCT/KR2024/004852, filed Apr. 11, 2024, which claims priority to Korean Patent Application No. 10-2023-0047532, filed Apr. 11, 2023, the contents of these applications being incorporated by reference herein in their entireties for all purposes.

### TECHNICAL FIELD

The present disclosure relates to a novel crystalline form of ceramide NP and a composition including the same.

### BACKGROUND OF THE INVENTION

Ceramides are mainly produced by the hydrolysis of sphingomyelin, which is a type of phospholipid that makes up cell membranes, by an enzyme called sphingomyelinase. Ceramides are a main polar lipid component of the stratum corneum, which accounts for about 50% of the stratum corneum of the skin, and is a component which forms a lipid bilayer with water to form a moisture-proof and barrier function.

Specifically, ceramides are basically based on a sphingolipid structure and have slightly different structures depending on the type thereof, such as having alkyl chains of different lengths or having different functional groups. For example, ceramide EOP (ceramide 1) has a structure including a long alkyl chain with 30 to 50 carbon atoms connected to an ether group, and ceramide NP (Ceramide 3) includes an alkyl chain with less than 30 carbon atoms.

Meanwhile, a compound may exist in one or more crystalline forms. Crystalline forms of a substance may have different chemical and physical properties, including melting point, chemical reactivity, solubility, rate of decomposition, optical and mechanical properties, vapor pressure, hygroscopicity, particle shape, density, flowability, and compatibility. These properties may directly affect the ability to process and/or manufacture the corresponding compound as a product. Crystalline forms may also exhibit different solubility or stability, etc.

Accordingly, the inventors of the present application completed a ceramide NP crystalline form with improved chemical and physical properties.

### SUMMARY OF THE INVENTION

One aspect is to provide a ceramide NP crystalline powder having a powder X-ray diffraction pattern including peaks at diffraction angles (2θ) of 4.607±0.20°, 6.92±0.20°, 11.531±0.20°, and 15.204±0.20°.

Another aspect is to provide a cosmetic composition including the ceramide NP crystalline powder.

Another aspect is to provide a composition for external preparation for the skin including the ceramide NP crystalline powder.

### BRIEF DESCRIPTION OF THE DRAWINGS

FIG. 1 shows an X-ray diffraction pattern of ceramide NP powder according to an embodiment.

FIG. 2 shows an X-ray diffraction pattern of ceramide NP of Comparative Example 1.

FIG. 3 shows the X-ray diffraction pattern of ceramide NP powder of Example 1 and the X-ray diffraction pattern of ceramide NP of Comparative Example 1 which are compared to each other by being arranged to overlap each other.

FIG. 4 shows a differential scanning calorimetry (DSC) pattern of ceramide NP powder according to an embodiment.

FIG. 5 shows a DSC pattern of ceramide NP of Comparative Example 1.

FIG. 6 shows DSC patterns of the ceramide NP powder according to an embodiment and the ceramide NP of Comparative Example 1 which are compared to each other by being arranged to overlap each other.

FIG. 7 shows thermogravimetric analysis (TGA), DSC, and Derivative thermogravity (DTG) patterns of ceramide NP powder according to an embodiment.

FIG. 8 shows the TGA, DSC, and DTG patterns of ceramide NP of Comparative Example 1.

FIG. 9 shows TGA, DSC, and DTG patterns of the ceramide NP powder according to an embodiment and the ceramide NP of Comparative Example 1 which are arranged to overlap each other to compare the same.

FIG. 10 shows the viscosity of Example 2 and Comparative Example 2 analyzed using a viscometer.

FIG. 11 shows the stability of Example 2 and Comparative Example 2 analyzed using a polarizing microscope

### DETAILED DESCRIPTION OF THE INVENTION

One aspect is to provide a ceramide NP crystalline powder having a powder X-ray diffraction pattern including peaks at diffraction angles (2θ) of 4.607±0.20°, 6.92±0.20°, 11.531±0.20°, and 15.204±0.20°.

In an embodiment, it is confirmed that the new crystalline form of the ceramide NP had new properties, which were characterized.

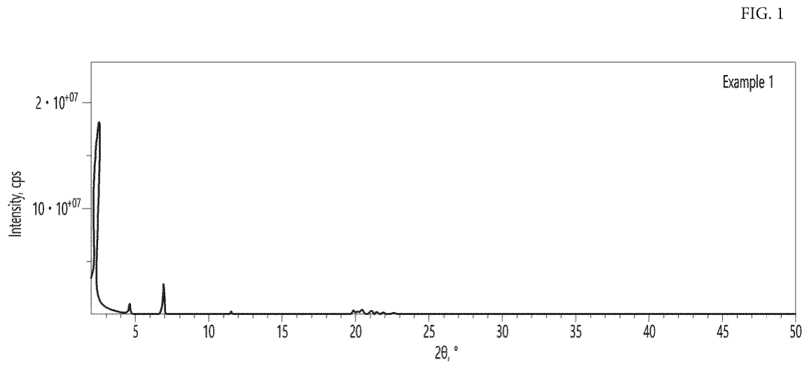

In an embodiment, the X-ray diffraction pattern of the ceramide NP crystalline powder is substantially as shown in FIG. 1.

In an embodiment, the ceramide NP crystalline powder may have an endothermic peak at about 80° C. to about 110° C. when measured by differential scanning calorimetry (DSC) at a temperature increase of 10° C./min.

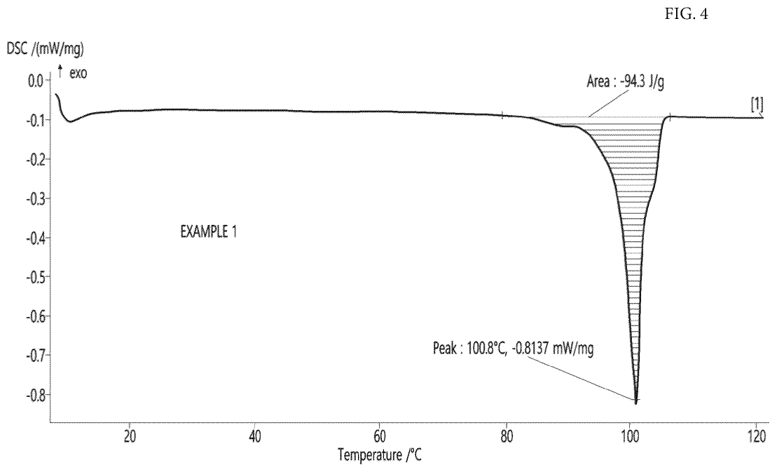

In an embodiment, the DSC graph of the ceramide NP crystalline powder is substantially as shown in FIG. 4.

In an embodiment, a weight loss of the ceramide NP crystalline powder at 260° C. during thermogravimetric analysis, is less than 5%.

In an embodiment, about 0.01% to about 10% of the ceramide NP crystalline powder may be a compound represented by Formula 1:

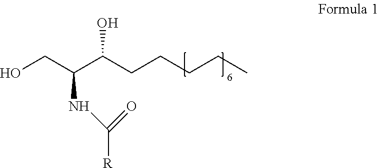

(R in Formula 1 is a linear or branched C₁₂-C₂₄ alkyl group, alkenyl group, or alkynyl group).

In some embodiments, about 0.01% to about 10%, about 0.01% to about 9%, about 0.01% to about 8%, about 0.01% to about 6%, about 0.01% to about 5%, about 0.01% to about 4%, about 0.01% to about 3%, about 0.05% to about 10%, about 0.05% to about 8%, about 0.05% to about 7%, about 0.05% to about 6%, about 0.05% to about 5%, about 0.05% to about 4%, about 0.05% to about 3%, about 0.1% to about 10%, about 0.1% to about 9%, about 0.1% to about 8%, about 0.1% to about 7%, about 0.1% to about 6%, about 0.1% to about 5%, about 0.1% to about 4%, about 0.1% to about 3%, about 0.5% to about 10%, about 0.5% to about 9%, about 0.5% to about 8%, about 0.5% to about 7%, about 0.5% to about 6%, about 0.5% to about 5%, about 0.5% to about 4%, or 0.5% to about 3% of the powder may be a compound represented by Formula 1. In this regard, the content may be based on wt %.

In an embodiment, about 90% to about 99.99% of the ceramide NP crystalline powder may be a compound represented by Formula 2:

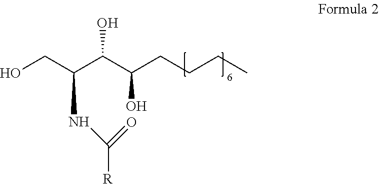

(R in Formula 2 may be a linear or branched C₁₂-C₂₄ alkyl group, alkenyl group, or alkynyl group).

In some embodiments, about 90% to about 99.99%, about 90% to about 99.5%, about 90% to about 99.0%, about 93% to about 99.99%, about 93% to about 99.5%, about 93% to about 99.0%, about 95% to about 99.99%, about 95% to about 99.5%, about 95% to about 99.0%, about 97% to about 99.99%, about 97% to about 99.5%, or 97% to about 99.0% of the powder may be the compound represented by Formula 2. In this regard, the content may be based on wt %.

The term “crystalline” as used herein refers to a crystalline solid that does not contain a substantially fixed molar proportion of solvent molecules within the crystal lattice, i.e., a crystalline solid that is not a solvate.

The term “characterization” as used herein refers to obtaining data that can be used to identify and distinguish the solid form of a compound, for example, whether the solid form is amorphous or crystalline and whether it is solvated or not. The process in which solid forms are characterized involves analyzing data collected from polymorphic forms to allow those skilled in the art to distinguish one solid form from other solid forms including the same substance. The chemical identity of solid forms may usually be determined by solution-state techniques for example, 13C NMR or 1H NMR. These can help identify materials, or solvent molecules for solvent compounds. However, such solution-state techniques themselves cannot provide information about the solid state. However, there are solid-state analytical techniques that can be used to provide information about solid-state structure and distinguish polymorphic solid forms, and examples of such techniques are single crystal X-ray diffraction, X-ray powder diffraction (XRPD), and solid-state nuclear magnetism resonance (SS-NMR) and infrared and Raman spectroscopy, and thermal techniques such as differential scanning calorimetry (DSC), thermogravimetric analysis (TGA), and melting point and high temperature microscopy.

To “characterize” a solid form of a compound, those skilled in the art may, for example, collect XRPD data on the solid forms of the compound and compare the XRPD peaks of the forms. For example, when two solid forms, that is, a first solid form and a second solid form, are compared, and Form 1 pattern exhibits a peak at an angle at which Form 2 pattern does not exhibit a peak, that peak for that compound may be used to distinguish Form 1 from Form 2 and further characterize Form 1. The collection of peaks that distinguish Form 1 from other known forms is the collection of peaks that can be used to characterize Form 1. Those skilled in the art will recognize that there are usually multiple methods for characterizing solid forms, including multiple methods using the same analytical technique. Additional peaks may also be used to characterize the morphology as much as possible, including the entire diffraction pattern. However, the additional peaks are optional. Although all peaks within the entire XRPD pattern can be used to characterize that shape, a subset of the data can be used to characterize the shape, and may be typically used.

The term “XRPD pattern” as used herein refers to an x-y graph with diffraction angle (typically °2θ) plotted on the x-axis and intensity plotted on the y-axis. Peaks within this pattern may be used to characterize the crystalline solid form. As with any data measurement, there is variability in XRPD data. Data are usually expressed solely by the diffraction angle of the peak rather than by the intensity of the peak. This is because peak intensity may be particularly sensitive to sample preparation (e.g. particle size, moisture content, solvent content and orientation effects affect sensitivity), and accordingly, samples of the same material prepared under different conditions may show slightly different patterns, and this variability is usually greater than the variability of the diffraction angle. Diffraction angle variability may also be sensitive to sample preparation. Other sources of variability are derived from instrument variables and the processing of raw X-ray data: different X-ray instruments operate using different variables, which can lead to slightly different XRPD patterns from the same solid form, and similarly, different software packages process X-ray data differently, which also results in variability. These and other causes of variability are known to those skilled in the art. Because of such sources of variability, a variability of ±0.2° 2θ is typically assigned to the diffraction angle in the XRPD pattern.

Another aspect is to provide a cosmetic composition including the ceramide NP crystalline powder.

Another aspect is to provide a composition for external preparation for the skin including the ceramide NP crystalline powder.

The crystalline powder is as described above.

The term “cosmetic composition” as used herein refers to an article used on the human body to clean and beautify the human body so as to add attractiveness and brighten the appearance, or maintain or promote the health of the skin and hair. The cosmetic composition may be used for improving skin condition.

In an embodiment, the improvement in skin condition may be skin moisturizing, strengthening the skin barrier, ameliorating skin aging, ameliorating skin wrinkles, improving skin elasticity, skin regeneration, or skin whitening.

The cosmetic composition may be prepared in any formulation commonly manufactured in the technical field to which the present disclosure pertains. For example, the formulation may be softening lotion, nourishing lotion, emulsion, lotion, cream, paste, gel, pack, solution, suspension, oil, wax, pack, powder, foundation, spray, hair product, surfactant-including cleansing, etc., but is not limited thereto. Examples of the formulation are skin lotion, skin softener, skin toner, nourishing cream, massage cream, milk lotion, powder, essence, eye cream, sun lotion, sunscreen, makeup primer, makeup base, BB cream, powder foundation, emulsion foundation, cleansing cream, cleansing foam, cleansing water, soap, pack, stick product, balm type product, spray, or powder.

The cosmetic composition may further include ingredients commonly used in cosmetics, such as thickeners, dispersants, fragrances, fillers, preservatives, antiseptics, neutralizers, sweeteners, vitamins, free-radical scavengers, metal ion sequestrants, functional ingredients, and mixtures thereof. Those skilled in the art will be able to select any additional ingredients and/or amounts thereof in such a manner that the advantageous properties of the compositions according to the present disclosure are not or substantially not adversely affected by the expected additions.

In an embodiment, the cosmetic composition may further include one or more types selected from the group consisting of preservatives, stabilizers, surfactants, thickeners, solubilizers, moisturizers, emollients, ultraviolet absorbers, antiseptics, disinfectants, emulsifiers, antioxidants, pH adjusters, organic and inorganic pigments, fragrances, cooling agents, and restricting agents.

In an embodiment, the cosmetic composition may have a formulation selected from the group consisting of skin, lotion, cream, essence, pack, foundation, a soap, a cleansing product, a body moisturizer, a hair product, and a detergent.

In an embodiment, the cosmetic composition may further include one or more selected from the group consisting of stabilizers, surfactants, oils, chelating agents, polyols, antiseptics, and thickeners.

The cosmetic composition may further include additional ingredients commonly used in cosmetics. Those skilled in the art will be able to select any additional ingredients and/or amounts thereof in such a manner that the advantageous properties of the compositions according to the present disclosure are not or substantially not adversely affected by the expected additions.

In this specification, the external preparation for the skin may be a cream, gel, ointment, skin emulsifier, skin suspension, transdermal delivery patch, drug-including bandage, lotion, or a combination thereof. The external preparation for the skin may be appropriately mixed according to need, with ingredients commonly used in external preparations for the skin in cosmetics and medicines, and examples of such ingredients are aqueous ingredients, oil-based ingredients, powder ingredients, alcohols, moisturizers, thickeners, ultraviolet absorbers, whitening agents, antiseptics, antioxidants, surfactants, and fragrances, colorants, various skin nutrients, or a combination thereof. The external preparation for the skin may be appropriately mixed with: metal sequestrants such as disodium edetate, trisodium edetate, sodium citrate, sodium polyphosphate, sodium metaphosphate, and gluconic acid; drugs such as caffeine, tannin, belafamil, licorice extract, glablidin, thermal water extract of calin fruit, various herbal medicines, tocopherol acetate, glytylitinic acid, tranexamic acid, and derivatives or salts thereof; vitamin C; magnesium ascorbate phosphate; or sugars such as ascorbic acid glucoside, arbutin, kojic acid, glucose, fructose, trehalose, etc.

The skin may include all skin areas of the body, including the face, hands, arms, legs, feet, chest, stomach, back, buttocks, and scalp.

The composition according to one aspect may be applied, administered, and applied to a subject. The “subject” as used herein refers to an object requiring antibacterial and disinfection, and more specifically, includes all mammals such as human or non-human primates, mice, dogs, cats, horses, and cows.

The cosmetic composition or the external preparation for the skin may be used singly or in multiple applications, or may be used in multiple applications with cosmetic compositions other than those of the present disclosure. In addition, the cosmetic composition according to the present disclosure may be used according to conventional usage methods, and the number of times of use may vary depending on the user's skin condition or preference.

Ceramide NP crystalline powder and compositions including the same, according to an aspect, have excellent stability properties and can be usefully used in compositions such as cosmetic compositions.

Each feature of the various experimental examples and embodiments of the present disclosure may be incorporated or combined with each other in part or in whole, and as will be fully appreciated by those skilled in the art, various interlocking and operational arrangements are technically possible, and each example and embodiment may be practiced independently of each other or together in association.

When interpreting components, it is interpreted to include the margin of error even if there is no separate explicit description.

The shapes, sizes, ratios, angles, numbers, etc. disclosed in the drawings for explaining experimental examples and embodiments of the present disclosure are provided for illustrative purpose, and the present disclosure is not limited to the matters shown. Additionally, in describing the present disclosure, if it is determined that a detailed description of related known technologies may unnecessarily obscure the gist of the present disclosure, the detailed description will be omitted. When ‘includes’, ‘has’, ‘consists of’, etc. described in this specification are used, other components may be added unless ‘only’ is used. In cases where a component is expressed in the singular, the plural is included unless specifically stated otherwise.

Hereinafter, it will be described in more detail through experimental examples and examples. However, these experimental examples and examples are intended to illustrate one or more specific examples and the scope of the present disclosure is not limited to these experimental examples and examples.

### EXAMPLES

### Example 1. Synthesis of Ceramide NP

Ceramide NP were synthesized through the following process.

Specifically, 5 g of Oleoyl chloride and 5 g of pyridine were dissolved in 20 ml of ethyl acetate and stirred. After 0.5 hours, 4 g of phytosphingosine prepared from Wickerhamomyces ciferrii SB8-25 (accession number KFCC11942P) was added thereto and caused to react at 50° C. for 2 hours, and then the reaction solution was cooled to 5° C., then subjected to filtration, obtaining ceramide NP in the form of white powder.

Hereinafter, experiments were performed to confirm the characteristics of the ceramide NP powder prepared in these examples, and as a comparative example, ceramide NP powder obtained from Matreya was used. The ceramide NP powder obtained from Matreya company may also be represented as Comparative Example 1.

### Experimental Example 1. Identification of Characteristics Through X-Ray Diffraction Analysis (XRD)

The crystal structure of the ceramide NP powder prepared in Example 1 was analyzed through X-ray diffraction (XRD) analysis.

X-ray diffraction patterns were collected with an X-ray diffractometer (X'Pert PRO MPD, PANalytical) using monochromated Cu Kα (1=0.1541 nm) radiation at 40 kV and 30 mA.

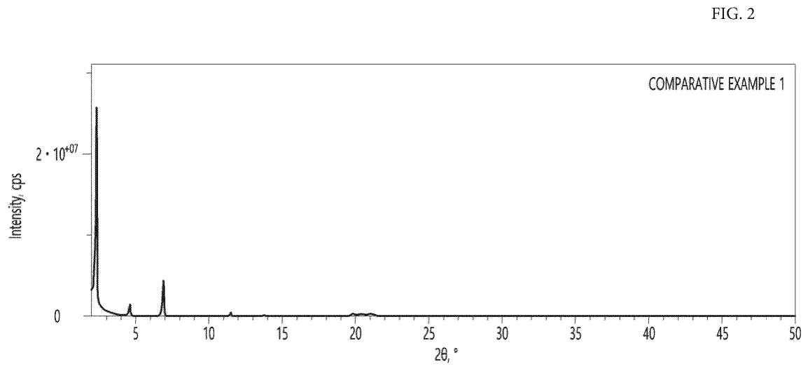

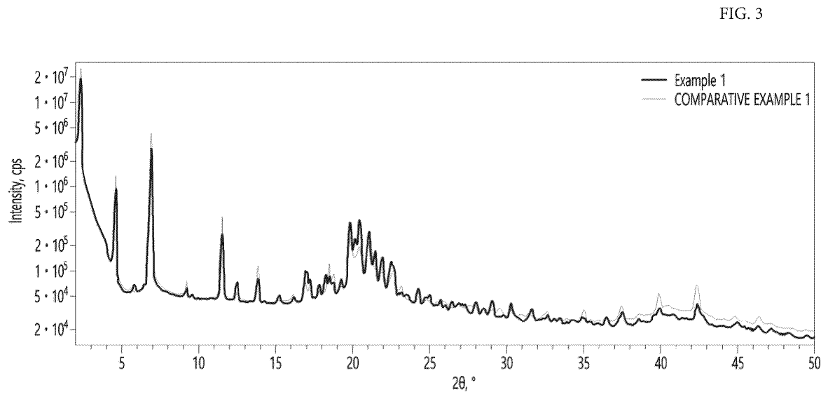

The X-ray diffraction pattern of the ceramide NP powder of Example 1 is shown in FIG. 1, and the X-ray diffraction pattern of the ceramide NP powder of Comparative Example 1 is shown in FIG. 2. The X-ray diffraction pattern of ceramide NP powder of Example 1 and the X-ray diffraction pattern of ceramide NP powder of Comparative Example 1 are compared to each other by being arranged to overlap each other in FIG. 3.

As shown in FIG. 1, the ceramide NP powder of Example 1 showed peaks at 2θ values of 4.607±0.20°, 6.92±0.20°, 11.531±0.20°, and 15.204±0.20°, and the ceramide NP powder of Comparative Example 1 showed peaks at 2θ values of 4.638±0.20°, 6.936±0.20°, and 11.540±0.20° As shown in FIG. 3, it was found that the diffraction pattern of Example 1 is different from that of the ceramide NP powder of Comparative Example 1. As described above, it was confirmed that the ceramide NP powder of Example 1 had different characteristics from the ceramide NP powder of Comparative Example 1.

### Experimental Example 2. Identification of Characteristics Through Differential Scanning Calorimetry (DSC)

DSC analysis was performed using an STA-1000 (Scinco, Korea) analyzer at 30° C. to 350° C. 4.32 mg of the ceramide NP powder of Example 1 was measured and added to an aluminum DSC pan, and non-hermetically sealed with a perforated aluminum lid, and the sample was heated from 20° C. to 120° C. at a scan rate of 10° C./min to monitor the generated heat flow reaction (DSC).

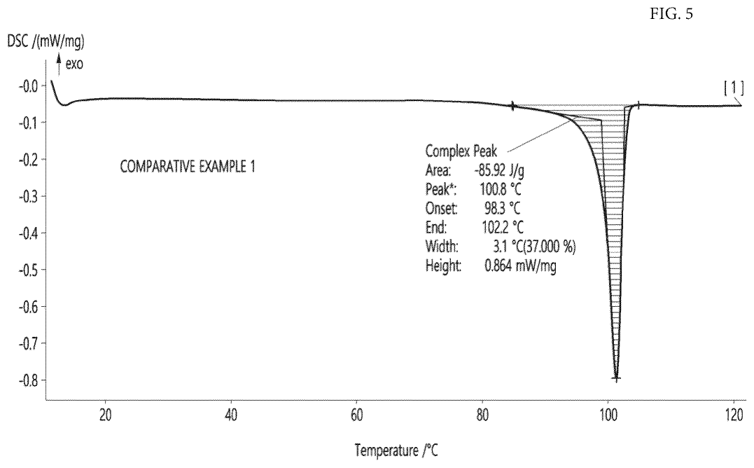

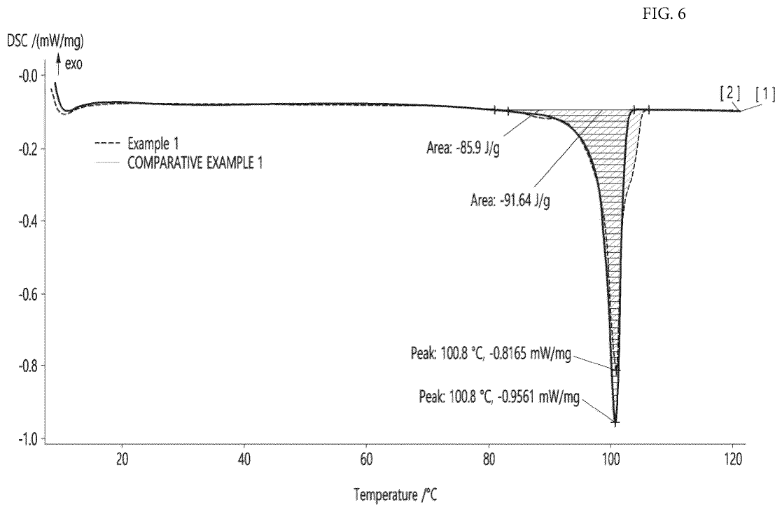

The DSC results of the ceramide NP powder of Example 1 are shown in FIG. 4, and the DSC results of the ceramide NP powder of Comparative Example 1 are shown in FIG. 5. The DSC results of the ceramide NP powder of Example 1 and the ceramide NP powder of Comparative Example 1 are compared to each other by being arranged to overlap each other in FIG. 6.

As shown in FIG. 4, the ceramide NP powder of Example 1 showed the maximum endothermic peak at 100.8° C. in DSC (10° C./min) having 85.1° C. as the starting point and 105.2° C. as the end point. As shown in FIG. 5, the ceramide NP powder of Comparative Example 1 showed the maximum endothermic peak at 100.8° C., with 86.4° C. as the starting point and 103.1° C. as the end point.

In addition, as shown in FIG. 6, it was found that the DSC result graph of the ceramide NP powder of Example 1 was different from the DSC result graph of the ceramide NP powder of Comparative Example 1. As described above, it was confirmed that the ceramide NP powder of Example 1 and the ceramide NP powder of Comparative Example 1 had different characteristics.

### Experimental Example 3. Identification of Characteristics Through Thermogravimetric Analysis (TGA)

Thermogravimetric analysis (TGA) was performed using a Mettler TGA 2 equipped with a 34 position auto-sampler. The instrument was calibrated for energy and temperature using certified Isoderm and Nickel. 10.01 mg of the ceramide NP powder of Example 1 was added to a pinhole aluminum pan and heated from 20° C. to 800° C. at 10° C./min to perform thermogravimetric analysis.

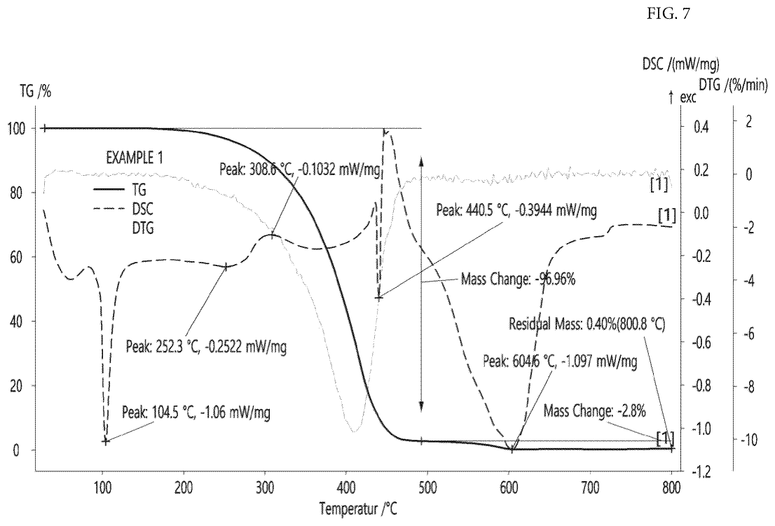

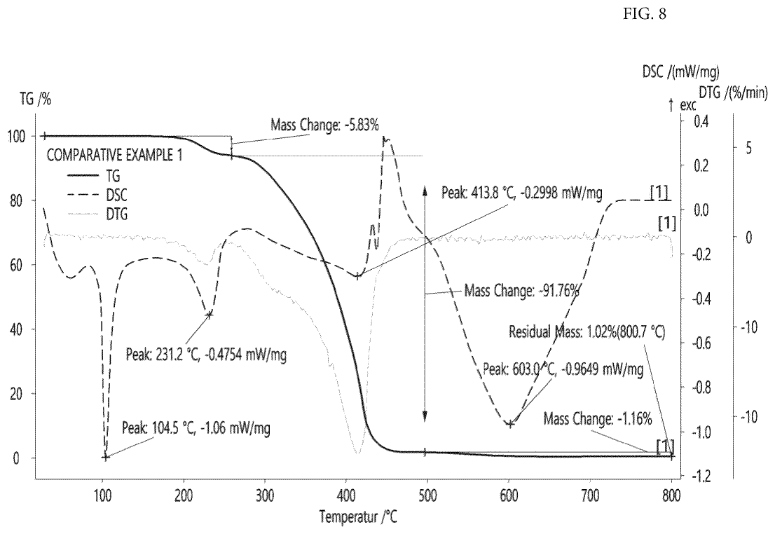

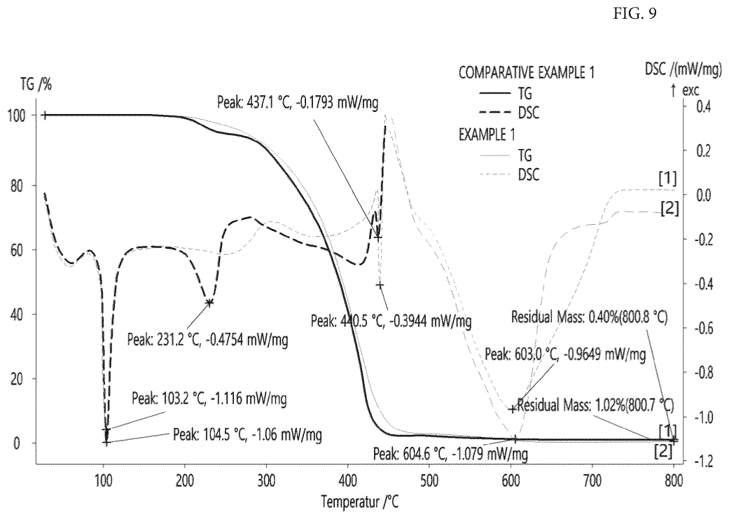

The TGA results of the ceramide NP powder of Example 1 are shown in FIG. 7, and the TGA results of the ceramide NP powder of Comparative Example 1 are shown in FIG. 8. The TGA results of ceramide NP powder of Example 1 and the ceramide NP powder of Comparative Example 1 are compared to each other by being arranged to overlap each other in FIG. 9.

As shown in FIG. 7, the ceramide NP powder of Example 1 showed no weight loss until decomposition began at 200° C. In addition, about 95% of the mass remained even at 268° C. without significant mass loss even when the temperature exceeded 200° C. As shown in FIG. 8, it was found that about 95% of the mass of the ceramide NP powder of Comparative Example 1 remained at 236° C.

In addition, as shown in FIG. 9, it was found that the TGA result graph of the ceramide NP powder of Example 1 is different from the TGA result graph of the ceramide NP powder of Comparative Example 1. As described above, it was confirmed that characteristics of the ceramide NP powder of Example 1 are different from characteristics of the ceramide NP powder of Comparative Example 1, and the ceramide NP powder of Example 1 is more stable than the ceramide NP powder of Comparative Example 1.

### Example 2 and Comparative Example 2. Preparation of Cream Using Ceramide NP

A cream that can be applied onto the human body was prepared by a conventional method according to the composition shown in Table 1 below, including the ceramide NP prepared in Example 1 and the ceramide NP prepared in Comparative Example 1. Specifically, Example 2 and Comparative Example 2 include the ceramide NP of Example 1 and the ceramide NP of Comparative Example 1, respectively. In this regard, the unit of content of each ingredient in Table 1 is wt %.

TABLE 1

| | | Comparative |
|---|---|---|
| | Example 2 | Example 2 |
| Ingredient name (product name) | (wt %) | (wt %) |
| Distilled water | 46.3 | 46.3 |
| Glycerin | 6.0 | 6.0 |
| Butylene glycol (1,3-BG) | 2.0 | 2.0 |
| Hyaluronic acid | 10.0 | 10.0 |
| Cetearyl olivate and sorbitan olivate (Olivem) | 1.0 | 1.0 |
| Carbomer (CP #941) | 15.0 | 15.0 |
| Ceramide NP | 0.5 | 0.5 |
| Glyceryl Monostearate (GMS 105) | 1.5 | 1.5 |
| Glyceryl Stearate/PEG-100 Stearate (Simulsol | 0.8 | 0.8 |
| #165) | | |
| C₁₄-C₂₂ alcohol, C₁₂-C₂₀ alkyl glucoside | 2.0 | 2.0 |
| (Montanov L) | | |
| Cetearyl alcohol (Lanette O) | 2.0 | 2.0 |
| Dimethicone (DC 200/100 CS) | 0.5 | 0.5 |
| Cyclopentalhexasiloxane (DC 345) | 2.0 | 2.0 |
| Cetyl ethyl hexanoate (CEH) | 3.0 | 3.0 |
| Hydrogenated polydecene (Puresyn 4) | 3.0 | 3.0 |
| Stearic acid | 0.8 | 0.8 |
| Arginine | 1.5 | 1.5 |
| 1,2-hexadiol (Hydrolite 6O) | 2.0 | 2.0 |
| Fragrance | 0.1 | 0.1 |

### Experimental Example 4. Cream Viscosity Measurement

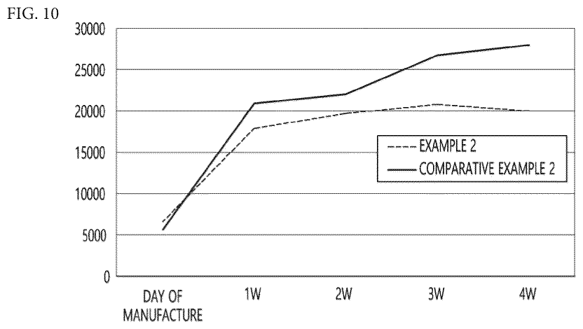

As shown in Table 1 above, the viscosity of the creams of Example 2 and Comparative Example 2 according to the type of ceramide NP powder was confirmed. Specifically, the viscosity (cP, centipoise) of the formulation was measured at room temperature for 1 minute using a spindle bar No. 5 at a rotation speed of 10 rpm with an LVT model viscometer (Brookfield engineering laboratories, Inc.). Results thereof are shown in FIG. 10.

As shown in FIG. 10, it was confirmed that the viscosity of Example 2 was maintained stably from the 2nd week, and the viscosity of Comparative Example 2 was continuously increased until the 4th week.

### Experimental Example 5. Identification of Crystal Structure Using a Polarizing Microscope

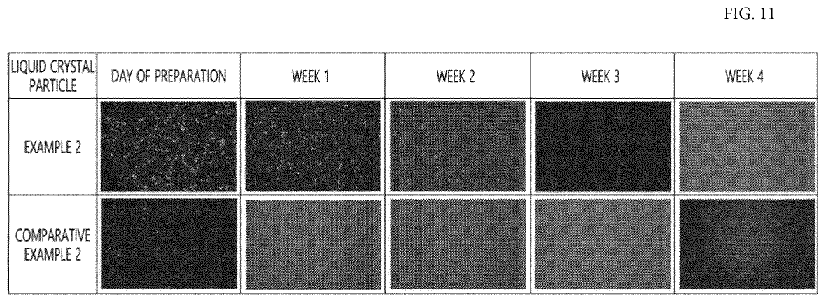

As shown in Table 1 above, the crystal structures of the creams of Example 2 and Comparative Example 2 according to the type of ceramide NP powder were identified. Specifically, liquid-crystal structures in the creams of Example 2 and Comparative Example 2 prepared above were identified at a magnification of ×400 using a polarizing microscope. Results thereof are shown in FIG. 11.

As shown in FIG. 11, it was confirmed that the liquid crystal structure of Example 2 was well maintained until the 4th week, and the liquid crystal structure of the cream of Comparative Example 2 was reduced after the 3rd week.

Through these results, it was confirmed that the ceramide NP crystalline powder of Example 1 of the present disclosure or the composition including the same, for example, the cream, has excellent stability characteristics.

1. A ceramide NP crystalline powder having a powder X-ray diffraction pattern including peaks at diffraction angles (2θ) of 4.607±0.20°, 6.92±0.20°, 11.531±0.20°, and 15.204±0.20°.

2. The ceramide NP crystalline powder of claim 1, having an endothermic peak at about 80° C. to about 110° C. when measured by differential scanning calorimetry (DSC) at a temperature increase of 10° C./min.

3. The ceramide NP crystalline powder of claim 1, wherein a weight loss thereof at 260° C. during thermogravimetrical analysis is less than 5%.

4. The ceramide NP crystalline powder of claim 1, wherein about 0.01% to about 10% of the ceramide NP crystalline powder is a compound represented by Formula 1: 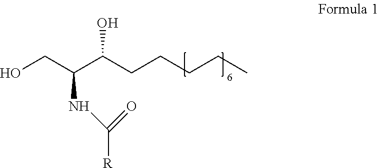
wherein R in Formula 1 is a linear or branched C₁₂-C₂₄ alkyl group, alkenyl group, or alkynyl group.

5. The ceramide NP crystalline powder of claim 1, wherein about 90% to about 99.99% of the ceramide NP crystalline powder is a compound represented by Formula 2: 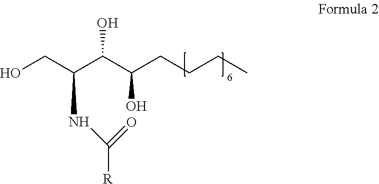
wherein R in Formula 2 is a linear or branched C₁₂-C₂₄ alkyl group, alkenyl group, or alkynyl group.

6. A cosmetic composition comprising the ceramide NP crystalline powder of claim 1.

7. The cosmetic composition of claim 6, further comprising one or more types selected from the group consisting of preservatives, stabilizers, surfactants, thickeners, solubilizers, moisturizers, emollients, ultraviolet absorbers, antiseptics, disinfectants, emulsifiers, antioxidants, pH adjusters, organic and inorganic pigments, fragrances, cooling agents, and restricting agents.

8. The cosmetic composition of claim 6, having a formulation selected from the group consisting of skin, lotion, cream, essence, pack, foundation, a soap, a cleansing product, a body moisturizer, a hair product, and a detergent.

9. An external preparation for a skin, comprising the ceramide NP crystalline powder of claim 1.
